(19) United States
(12) Patent Application Publication
(10) Pub. No.: US 2024/0029889 A1
(43) Pub. Date: Jan. 25, 2024

(54) MACHINE LEARNING-BASED DISEASE DIAGNOSIS AND TREATMENT

(71) Applicants: Nima Sharifi Sadeghi, Tehran (IR); Sayyed Hassan Saadat Mirghadim, Lahijan (IR); Mohammad Bagher Menhaj, Tehran (IR)

(72) Inventors: Nima Sharifi Sadeghi, Tehran (IR); Sayyed Hassan Saadat Mirghadim, Lahijan (IR); Mohammad Bagher Menhaj, Tehran (IR)

(73) Assignees: Nima Sahrifi Sadeghi, Tehran, OT (IR); Sayyed Hassan Saadat Mirghadim, Lahijan, OT (IR); Mohammad Bagher Menhaj, Tehran, OT (IR); Amirkabir University of tech.Amirkabir University of tech.Amirkabir University of tech., Tehran, OT (IR)

(21) Appl. No.: 18/357,935

(22) Filed: Jul. 24, 2023

Related U.S. Application Data
(63) Continuation-in-part of application No. PCT/IB2021/060788, filed on Nov. 21, 2021.
(60) Provisional application No. 63/140,858, filed on Jan. 24, 2021.

(51) Int. Cl. G16H 50/20; G16H 20/00; G16H 10/60

(52) U.S. Cl. CPC G16H 50/20; G16H 20/00; G16H 10/60

(57) ABSTRACT

A method for machine learning-based disease diagnosis and treatment of a subject. The method includes obtaining a clinical data set, obtaining a para-clinical data set, detecting a first status by applying a first classifier to the clinical data set, detecting a second status by applying a second classifier to the para-clinical data set, detecting a final status by applying a first ensemble model to the first status and the second status, and determining a treatment plan of the subject based on the final status. The clinical data set is associated with clinical symptoms of the subject. The para-clinical data set includes at least one of a plurality of medical images, a plurality of biomedical signals, and a plurality of para-clinical test results of the subject. Each of the first status, the second status, and the final status representing one of illness or healthiness of the subject.

### CROSS-REFERENCE TO RELATED APPLICATION

This application is a continuation-in-part of International Patent Application PCT/IB2021/060788, filed on Nov. 21, 2021, and entitled “DISEASE DIAGNOSIS BASED ON CLINICAL AND PARA-CLINICAL DATA,” which takes priority from U.S. Provisional Patent Application Ser. No. 63/140,858, filed on Jan. 24, 2021, and entitled “INTELLIGENT CLOUD SYSTEMS FOR EARLY DETECTION OF DISEASE, DRUG INTERACTIONS, AND DIAGNOSIS OF NEUROLOGICAL DISEASES USING DEEP NEURAL NETWORKS,” which are all incorporated herein by reference in their entirety.

### TECHNICAL FIELD

The present disclosure generally relates to medical diagnosis, and particularly, medical diagnosis based on machine learning methods.

### BACKGROUND

Medical diagnosis is a process of determining a condition of a disease based on a subject's symptoms and signs. A health status obtained from a medical diagnosis determines medical decisions about possible treatment. Required information for diagnosis is typically collected from clinical symptoms of a subject. Detecting health status is often challenging, because many symptoms are nonspecific. For example, hypertension may be a symptom of many disorders. Thus, a healthcare professional may need some complementary information to correctly diagnose a disease. Therefore, one or more medical tests are also performed to provide a healthcare professional with medical images, biomedical signals, and medical test results of a subject.

Conventional diagnostic procedures include obtaining diagnostic information and interpretation of information by healthcare physicians. In many cases, different pieces of diagnostic information may require to be interpreted by a respective specialist. For example, a radiologist may interpret medical images and provide a neurologist with a report including possible abnormalities in a subject's brain. Therefore, conventional diagnostic procedures may be slow and subjected to human errors. Intelligent diagnostic methods may facilitate and expedite a diagnostic procedure, while reducing diagnostic errors. However, conventional intelligent diagnostic methods make use of a single type of diagnostic information, ignoring other pieces of diagnostic information. In some conventional methods, only medical images obtained by computed tomography scan or magnetic resonance imaging are analyzed to determine a health status. Other methods only process biomedical signals such as electroencephalography or evoked potential signals. Apart from medical images and biomedical signals, clinical test results are also used to determine a health status. However, health status that detected only based on a single type of data may not provide sufficient precision of diagnosis.

There is, therefore, a need for a health status detection method based on artificial intelligence that utilizes various diagnostic information of a subject such as clinical symptoms, medical images, biomedical signals, and para-clinical test results.

### SUMMARY

This summary is intended to provide an overview of the subject matter of the present disclosure, and is not intended to identify essential elements or key elements of the subject matter, nor is it intended to be used to determine the scope of the claimed implementations. The proper scope of the present disclosure may be ascertained from the claims set forth below in view of the detailed description below and the drawings.

In one general aspect, the present disclosure describes an exemplary method for machine learning-based disease diagnosis and treatment of a subject. An exemplary method may include obtaining a clinical data set, obtaining a para-clinical data set, detecting a first status based on the clinical data set, detecting a second status based on the para-clinical data set, detecting a final status based on the first status and the second status, and determining a treatment plan of the subject based on the final status. An exemplary clinical data set may be associated with clinical symptoms of the subject. In an exemplary embodiment, obtaining the para-clinical data set may include at least one of obtaining a plurality of medical images from the subject, obtaining a plurality of biomedical signals from the subject, and obtaining a plurality of para-clinical test results of the subject. An exemplary plurality of medical images may be obtained utilizing a set of imaging devices. An exemplary plurality of biomedical signals may be obtained utilizing a set of biomedical signal acquisition devices. An exemplary first status may be detected utilizing one or more processors. An exemplary first status may be detected by applying a first classifier to the clinical data set. An exemplary second status may be obtained utilizing the one or more processors. An exemplary second status may be detected by applying a second classifier to the para-clinical data set. An exemplary final status may be obtained utilizing the one or more processors. An exemplary final status may be detected by applying a first ensemble model to the first status and the second status. In an exemplary embodiment, each of the first status, the second status, and the final status may represent one of illness or healthiness of the subject.

In an exemplary embodiment, applying the first ensemble model may include generating a first decision value based on the first status and the second status, setting a bias of each activation function of a multi-layer perceptron to a first bias value, setting the bias to a second bias value, and applying the multi-layer perceptron to the first status and the second status. In an exemplary embodiment, generating the first decision value may include applying a decision rule to the first status and the second status. An exemplary bias may be set to the first bias value responsive to the first decision value being larger than or equal to a decision threshold. An exemplary bias may be set to the second bias value responsive to the first decision value being smaller than the decision threshold.

In an exemplary embodiment, applying the first classifier to the clinical data set may include detecting a first plurality of status based on the clinical data set and generating the first status based on the first plurality of status. In an exemplary embodiment, detecting the first plurality of status may include applying each of a plurality of statistical processes to a respective subset of the clinical data set. In an exemplary embodiment, each subset of the clinical data set may be associated with a respective clinical examination. In an exemplary embodiment, generating the first status may include applying a second ensemble model to the first plurality of status. In an exemplary embodiment, each of the first plurality of status may include a respective binary value representing one of the illness or the healthiness

In an exemplary embodiment, applying the second ensemble model to the first plurality of status may include detecting a second plurality of status from the clinical data set, detecting a third plurality of status from the second plurality of status, and applying the second ensemble model to the third plurality of status. In an exemplary embodiment, detecting the second plurality of status may include applying each of a plurality of adaptive neuro fuzzy inference systems to a respective subset of the clinical data set. In an exemplary embodiment, each of the second plurality of status may include a respective binary value representing one of the illness or the healthiness. In an exemplary embodiment, detecting the third plurality of status may include applying each of a plurality of ensemble models to a respective status of the first plurality of status and a respective status of the second plurality of status. In an exemplary embodiment, each of the third plurality of status may include a respective binary value representing one of the illness or the healthiness.

In an exemplary embodiment, applying the second classifier to the para-clinical data set may include detecting a fourth plurality of status from the plurality of medical images and detecting an image status from the fourth plurality of status. In an exemplary embodiment, detecting the fourth plurality of status may include applying a first plurality of machine learning-based classifiers to the plurality of medical images. In an exemplary embodiment, detecting the image status may include applying a third ensemble model to the fourth plurality of status.

In an exemplary embodiment, the first plurality of machine learning-based classifiers may include a plurality of U-Nets. In an exemplary embodiment, the plurality of medical images may include at least one of computed tomography (CT) scan images, magnetic resonance imaging (MRI) images, magnetic resonance venography (MRV) images, magnetic resonance spectroscopy (MRS) images, and positron-emission tomography (PET) images. An exemplary image status may represent one of the healthiness or being subjected to a multiple sclerosis (MS) disease.

In an exemplary embodiment, applying the second classifier to the para-clinical data set may further include detecting a fifth plurality of status from the plurality of biomedical signals and detecting a biomedical status from the fifth plurality of status. In an exemplary embodiment, detecting the fifth plurality of status may include applying a second plurality of machine learning-based classifiers to the plurality of biomedical signals. In an exemplary embodiment, detecting the biomedical status may include applying a fourth ensemble model to the fifth plurality of status.

An exemplary second plurality of machine learning-based classifiers may include a k-nearest neighbors classifier, a support vector machine classifier, and a recurrent neural network. An exemplary plurality of biomedical signals may include an auditory evoked potential, a somatosensory evoked potential, a visually evoked potential, an electroretinogram signal, an electroneurogram signal, an electromyogram signal, and an electroencephalography signal.

In an exemplary embodiment, applying the second classifier to the para-clinical data set may further include detecting a sixth plurality of status from the plurality of para-clinical test results and detecting a para-clinical status from the sixth plurality of status. In an exemplary embodiment, detecting the sixth plurality of status may include comparing the plurality of para-clinical test results with a plurality of threshold values. In an exemplary embodiment, detecting the para-clinical status may include applying a fifth ensemble model to the sixth plurality of status.

In an exemplary embodiment, comparing the plurality of para-clinical test results with the plurality of threshold values may include comparing each test result of a plurality of lumbar puncture test results and a plurality of blood test results with a respective threshold value of the plurality of threshold values.

In an exemplary embodiment, each of the third ensemble model, the fourth ensemble model, and the fifth ensemble model may include a respective majority voting model. In an exemplary embodiment, applying the second classifier to the para-clinical data set may further include applying a bootstrap aggregation model to the image status, the biomedical status, and the para-clinical status.

In an exemplary embodiment, applying the second classifier to the para-clinical data set may further include calculating a weighted average, detecting a third status, setting the second status to a second decision value based on the weighted average and the third status, and setting the second status to a third decision value based on the weighted average and the third status. In an exemplary embodiment, calculating the weighted average may include calculating a weighted average of the image status, the biomedical status, and the para-clinical status. In an exemplary embodiment, detecting the third status may include applying a neural network-based classifier to the image status, the biomedical status, and the para-clinical status. An exemplary second status may be set to the second decision value responsive to each of the weighted average and the third status being equal to the second decision value. An exemplary second status may be set to the third decision value responsive to the weighted average and the third status being different.

Other exemplary systems, methods, features and advantages of the implementations will be, or will become, apparent to one of ordinary skill in the art upon examination of the following figures and detailed description. It is intended that all such additional systems, methods, features and advantages be included within this description and this summary, be within the scope of the implementations, and be protected by the claims herein.

### BRIEF DESCRIPTION OF THE DRAWINGS

The drawing figures depict one or more implementations in accord with the present teachings, by way of example only, not by way of limitation. In the figures, like reference numerals refer to the same or similar elements.

FIG. 1A shows a flowchart of a method for machine learning-based disease diagnosis of a subject, consistent with one or more exemplary embodiments of the present disclosure.

FIG. 1B shows a flowchart of a method for obtaining a para-clinical data set, consistent with one or more exemplary embodiments of the present disclosure.

FIG. 1C shows a flowchart for obtaining a first status, consistent with one or more exemplary embodiments of the present disclosure.

FIG. 1D shows a flowchart for applying an ensemble model to a plurality of status, consistent with one or more exemplary embodiments of the present disclosure.

FIG. 1E shows a flowchart for applying a classifier to a para-clinical data set, consistent with one or more exemplary embodiments of the present disclosure.

FIG. 1F shows a flowchart for applying a classifier to a number of status, consistent with one or more exemplary embodiments of the present disclosure.

FIG. 1G shows a flowchart for applying an ensemble model to a first status and a second status, consistent with one or more exemplary embodiments of the present disclosure.

FIG. 2A shows a schematic of a system for disease diagnosis, consistent with one or more exemplary embodiments of the present disclosure.

FIG. 2B shows a schematic of a first classifier, consistent with one or more exemplary embodiments of the present disclosure.

FIG. 2C shows a schematic of a second classifier, consistent with one or more exemplary embodiments of the present disclosure.

FIG. 2D shows a schematic of an ensemble model, consistent with one or more exemplary embodiments of the present disclosure.

FIG. 2E shows a schematic of an ensemble model detecting a final status, consistent with one or more exemplary embodiments of the present disclosure.

FIG. 3 shows a high-level functional block diagram of a computer system, consistent with one or more exemplary embodiments of the present disclosure.

FIG. 4 shows magnetic resonance images of a subject's brain, consistent with one or more exemplary embodiments of the present disclosure.

FIG. 5 shows electroencephalography signals of a subject, consistent with one or more exemplary embodiments of the present disclosure.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

The following detailed description is presented to enable a person skilled in the art to make and use the methods and devices disclosed in exemplary embodiments of the present disclosure. For purposes of explanation, specific nomenclature is set forth to provide a thorough understanding of the present disclosure. However, it will be apparent to one skilled in the art that these specific details are not required to practice the disclosed exemplary embodiments. Descriptions of specific exemplary embodiments are provided only as representative examples. Various modifications to the exemplary implementations will be readily apparent to one skilled in the art, and the general principles defined herein may be applied to other implementations and applications without departing from the scope of the present disclosure. The present disclosure is not intended to be limited to the implementations shown, but is to be accorded the widest possible scope consistent with the principles and features disclosed herein.

Herein is disclosed an exemplary method and system for detecting health status of a subject. An exemplary method may detect a health status about a subject's disease based on both clinical data and para-clinical data of the subject. An exemplary method may detect a clinical status by analyzing clinical symptoms of a subject. Different sets of clinical symptoms may be obtained from standard medical tests and separately processed by adaptive neuro-fuzzy inference systems (ANFISs) and statistical methods such as hypothesis testing. Then, a clinical status may be detected by applying a majority voting to different outputs of hypothesis testing. Besides, an exemplary para-clinical status may be detected from medical images, biomedical signals, and para-clinical test results. Medical images and biomedical signals may be classified by a number of exemplary machine learning-based classifiers, and respective status may be detected by applying respective majority voting models to detected classes of medical images and biomedical signals. In addition, another status may be detected by classifying para-clinical test results. Then, an exemplary para-clinical status may be detected by applying an ensemble model such as bootstrap aggregation to different classes that are detected from medical images, biomedical signals, and para-clinical test results. Finally, a status may be detected by applying an exemplary ensemble model such as boosting method to both clinical status and para-clinical status.

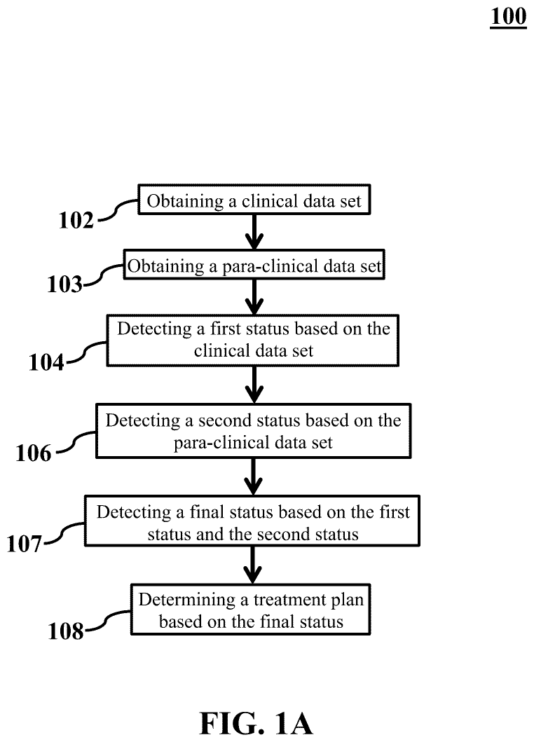

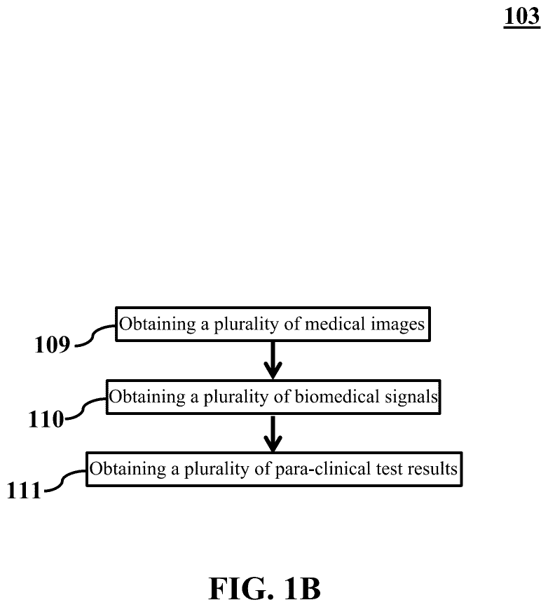

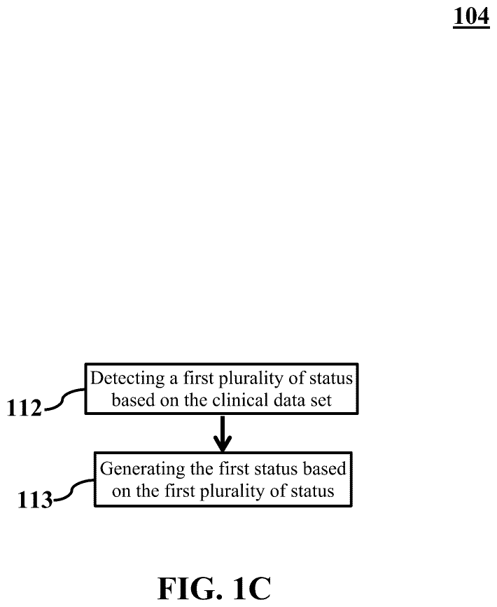

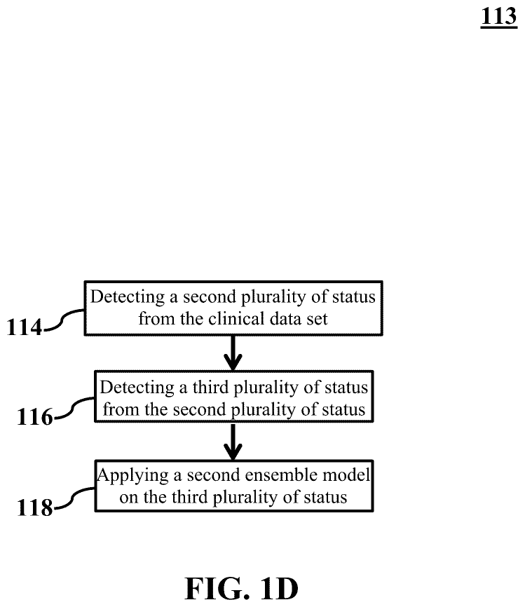

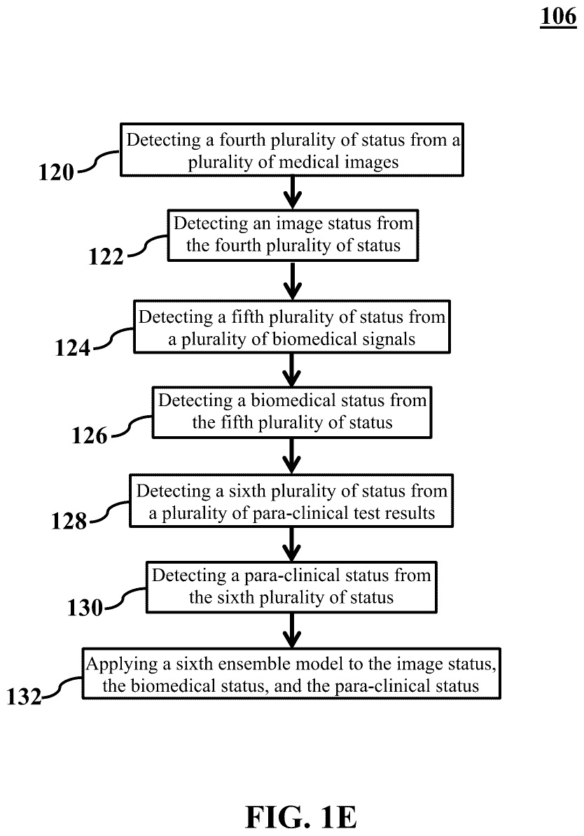

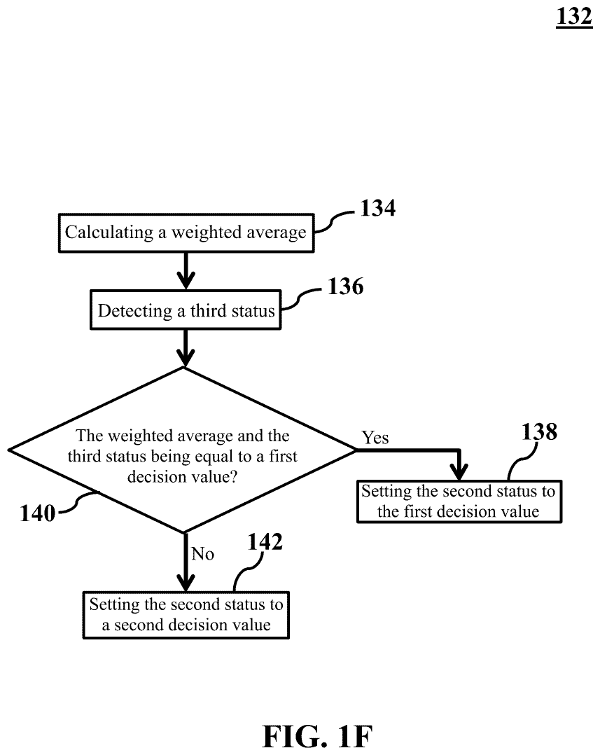

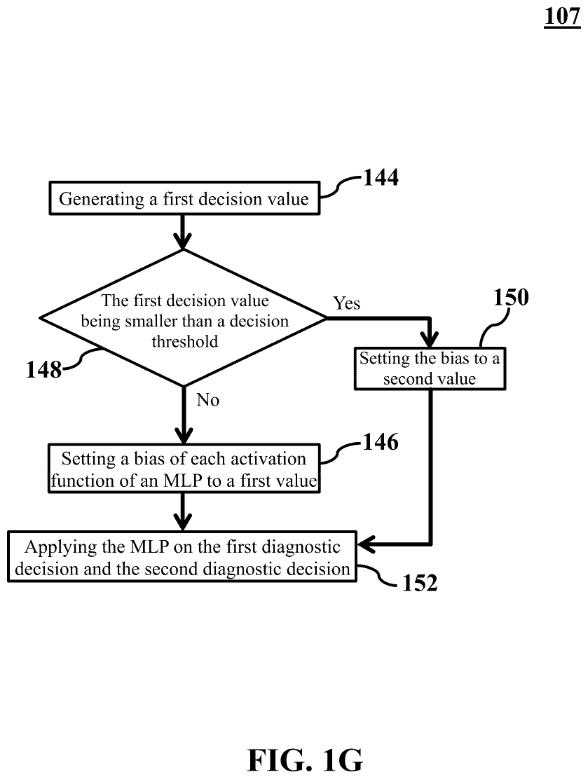

FIG. 1A shows a flowchart of a method for machine learning-based disease diagnosis and treatment of a subject, consistent with one or more exemplary embodiments of the present disclosure. An exemplary method 100 may include obtaining a clinical data set (step 102), obtaining a para-clinical data set (step 103), detecting a first status based on the clinical data set (step 104), detecting a second status based on the para-clinical data set (step 106), detecting a final status based on the first status and the second status (step 107), and determining a treatment plan of the subject (step 108). In an exemplary embodiment, method 100 may provide a diagnostic decision about a status of a disease. An exemplary status may determine either a subject is healthy or not. An exemplary status may also determine a severity of a subject's disease. In an exemplary embodiment, each of the first status, the second status, and the final status may represent one of illness or healthiness of the subject.

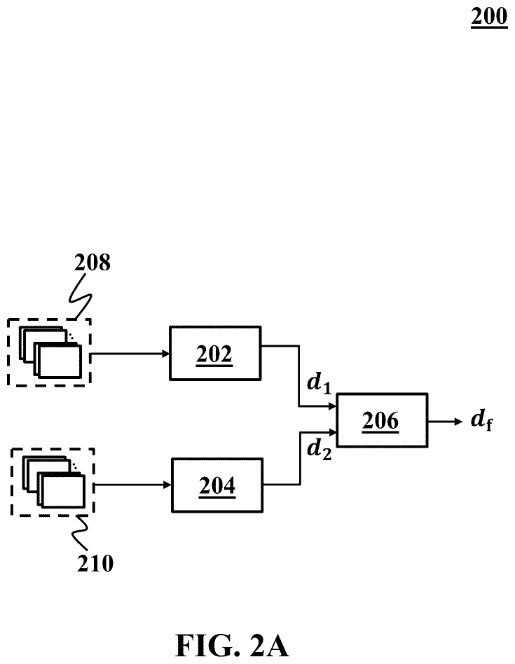

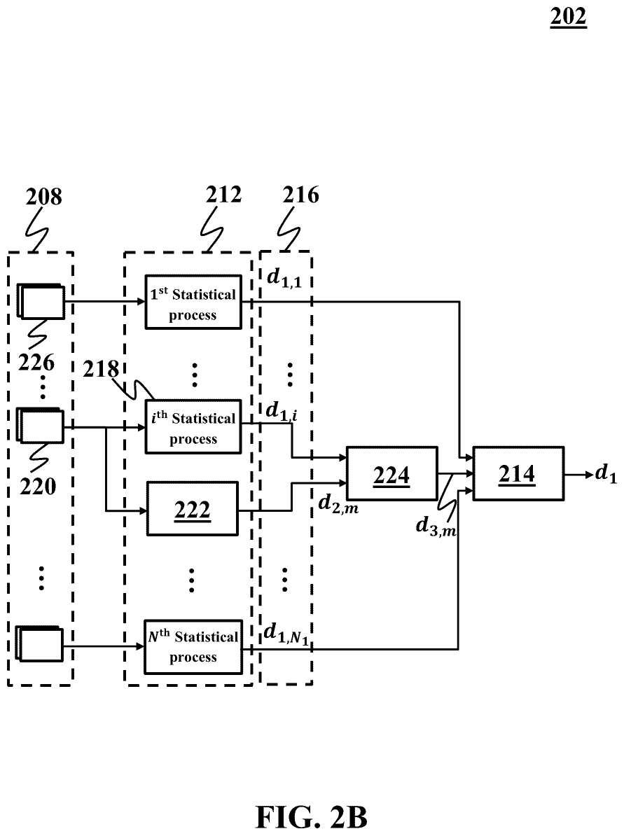

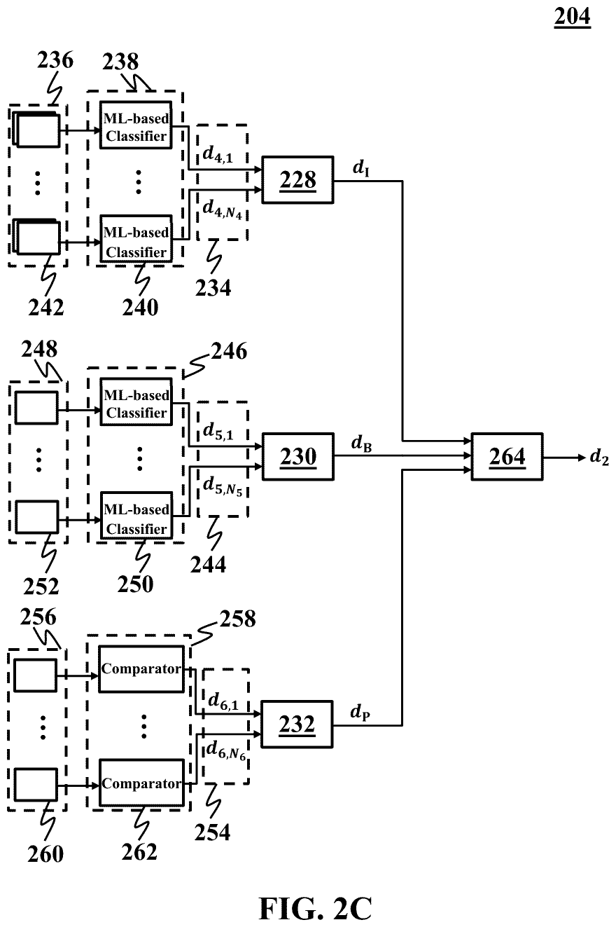

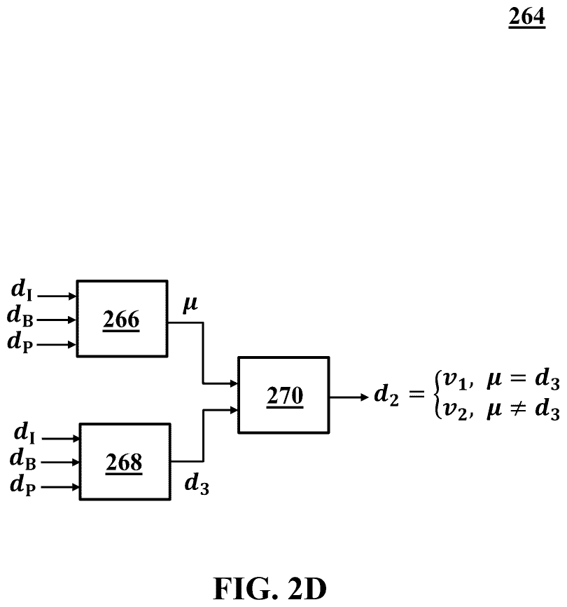

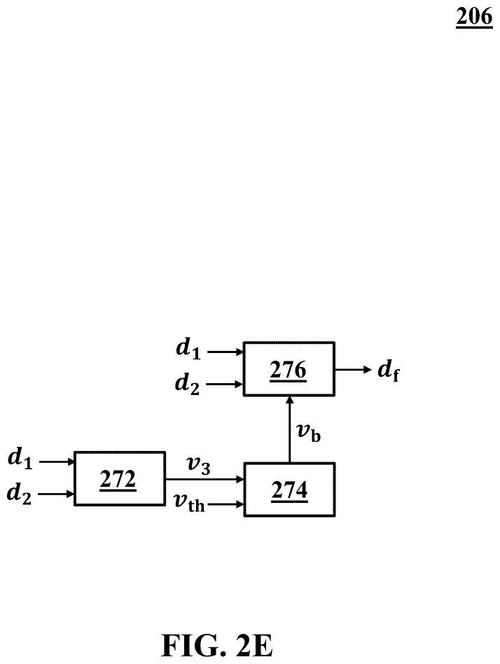

FIG. 2A shows a schematic of a system for disease diagnosis, consistent with one or more exemplary embodiments of the present disclosure. In an exemplary embodiment, different steps of method 100 may be implemented utilizing a system 200. In an exemplary embodiment, system 200 may include a first classifier 202, a second classifier 204, and a first ensemble model 206. First classifier 202, second classifier 204, and first ensemble model 206 may be implemented utilizing an exemplary processor. In an exemplary embodiment, each of first classifier 202, second classifier 204, and first ensemble model 206 may be implemented by a respective machine learning model.

Referring to FIGS. 1A and 2A, in an exemplary embodiment, step 102 may include obtaining a clinical data set 208. In an exemplary embodiment, clinical data set 208 may be associated with clinical symptoms of a subject. In an exemplary embodiment, clinical data set 208 may be obtained by physical examination of a subject. In an exemplary embodiment, clinical data set 208 may be obtained from standard medical tests. An exemplary medical test may include a number of questions about symptoms of a disease. A subject may answer a number of exemplary standard tests. In an exemplary embodiment, each subset of clinical data set 208 may be associated with a respective clinical examination. In an exemplary clinical examination, a healthcare professional may examine a subject for possible medical signs or symptoms of a medical condition. An exemplary clinical examination may include a series of questions about a subject's medical history followed by an examination. In an exemplary embodiment, clinical data set 208 may be obtained by a healthcare professional. In an exemplary embodiment, the healthcare professional may examine a subject for symptoms of a neurological disease such as a multiple sclerosis (MS) disease. Besides, a number of exemplary clinical tests may be taken from a subject and answers to each test may include a respective subset of clinical data set 208. Specifically, clinical data set 208 may be obtained by storing answers of a subject to a clinical questionnaire, that is, a clinical test, into an exemplary memory. Stored clinical data set 208 in the memory may then be transferred to an exemplary processor for applying a number of statistical processes to clinical data set 208. Exemplary clinical tests for MS diagnosis may include a McDonald test, an expanded disability status scale (EDSS) test, a 36-item short form health survey (SF-36) test, a bowel control scale (BWCS) test, an intravenous immunoglobulin (IVIG) test, a modified form of the fatigue impact scale (MFIS) test, a mental health inventory (MHI) test, a perceived deficits questionnaire (PDQ) test, a sexual satisfaction scale (SSS) test, a bladder control scale (BLCS) test, a Snellen test, a multiple sclerosis quality of life-54 (MSQOL-54) test, and a multiple sclerosis functional composite (MSFC) test. Each exemplary test may generate a respective score, indicating a status about severity of a subject's MS.

FIG. 1B shows a flowchart of a method for obtaining a para-clinical data set, consistent with one or more exemplary embodiments of the present disclosure. Referring to FIGS. 1B and 2A, in an exemplary embodiment, step 103 may include obtaining a para-clinical data set 210 by at least one of obtaining a plurality of medical images (step 109), obtaining a plurality of biomedical signals (step 110), and obtaining a plurality of para-clinical test results of the subject (step 111).

For further detail with respect to step 109, in an exemplary embodiment, the plurality of medical images may be obtained utilizing a set of imaging devices. Exemplary medical images may be obtained by magnetic resonance imaging, computer tomography scan, and positron-emission tomography. For obtaining an exemplary medical image, a subject may be exposed to an imaging device and a healthcare professional may capture a medical image utilizing the imaging device. Then, the medical image may be transferred to an exemplary processor for applying a machine learning-based classifier to the medical image.

In further detail with regard to step 110, in an exemplary embodiment, the plurality of biomedical signals may be obtained utilizing a set of biomedical signal acquisition devices. Exemplary biomedical signals may be obtained from brain or muscles of a subject. For obtaining an exemplary biomedical signal, a subject may be exposed to a biomedical signal acquisition device and a healthcare professional may capture a biomedical signal utilizing the biomedical signal acquisition device. A healthcare professional may obtain an exemplary EEG signal by placing a number of EEG electrodes on a subject's scalp and recording a data set of EEG electrodes. A healthcare professional may obtain an exemplary ECG signal by applying a number of ECG electrodes to a subject's body and recording a data set of ECG electrodes. Then, biomedical signals such as the EEG signal and the ECG signal may be transferred to an exemplary processor for applying a number of machine learning-based classifiers to biomedical signals.

For further detail regarding step 111, in an exemplary embodiment, a plurality of para-clinical test results may be obtained by para-clinical examination of the subject. In an exemplary embodiment, plurality of para-clinical test results may include a plurality of lumbar puncture test results and a plurality of blood test results. For obtaining an exemplary lumbar puncture test result, a healthcare professional may insert a needle into a space between two lumbar bones of a subject to remove a sample of cerebrospinal fluid. Then, an exemplary lumbar puncture test result may be obtained by examining the sample of cerebrospinal fluid for levels of white blood cells, neurofilaments, and oligoclonal bands. For obtaining an exemplary blood test result, a healthcare professional may take a blood sample from a subject and examine the blood sample for levels of vitamins and minerals.

Referring again to FIG. 1A, in an exemplary embodiment, step 104 may include detecting the first status. For further detail with respect to step 104, FIG. 1C shows a flowchart for detecting a first status, consistent with one or more exemplary embodiments of the present disclosure. Referring to FIGS. 1C and 2A, in an exemplary embodiment, a first status d₁ may be detected by applying first classifier 202 to clinical data set 208. First status d₁ may be detected by first classifier 202 utilizing an exemplary processor. In an exemplary embodiment, applying first classifier 202 to clinical data set 208 may include detecting a first plurality of status based on clinical data set 208 (step 112) and generating first status d₁ based on the first plurality of status (step 113).

FIG. 2B shows a schematic of a first classifier, consistent with one or more exemplary embodiments of the present disclosure. In an exemplary embodiment, first classifier 202 may include a plurality of statistical processes 212 and a second ensemble model 214. In what follows, an exemplary arrangement of plurality of statistical processes 212 and second ensemble model 214 in first classifier 202 is described as follows.

Referring to FIGS. 1B and 2B, in an exemplary embodiment, step 112 may include detecting a first plurality of status 216. In an exemplary embodiment, each of the first plurality of status may include a respective binary value representing one of the illness or the healthiness of the subject. In an exemplary embodiment, a status d_{1,i} of first plurality of status 216 may be detected by applying an i^{th} statistical process 218 of plurality of statistical processes 212 to an i^{th} subset 220 of clinical data set 208. In an exemplary embodiment, each statistical process of plurality of statistical processes 212 may detect a respective status by applying a hypothesis testing to a subject's answers to each clinical test. In an exemplary hypothesis testing, a probability may be computed for each of two hypotheses corresponding to illness or healthiness of a subject. An exemplary probability may be obtained from answers of a subject to each test. An exemplary status may include a value regarded to health of a subject when a probability with hypothesis corresponding to illness of the subject is smaller than a significance level. In contrast, an exemplary status may include a value regarded to illness of a subject when a probability with hypothesis corresponding to health of the subject is smaller than a significance level. An exemplary significance level may include one of 0.05 or 0.01. In an exemplary embodiment, a subject may take an EDSS test under administration of a healthcare professional. An exemplary EDSS test may include a number of EDSS scores ranging from 0 to 5. An exemplary hypothesis testing may be applied to EDSS scores, and then, an illness or healthiness of the subject may be detected from a p-value of the hypothesis testing. Specifically, when an exemplary p-value obtained from EDSS scores under an illness hypothesis is less than 0.05, the subject is regarded as healthy.

For further detail with respect to step 113, FIG. 1D shows a flowchart for applying an ensemble model to a plurality of status, consistent with one or more exemplary embodiments of the present disclosure. Referring to FIGS. 1D and 2B, in an exemplary embodiment, step 113 may include generating first status d₁ based on first plurality of status 216. In an exemplary embodiment, generating first status d₁ may include applying second ensemble model 214 to first plurality of status 216. In an exemplary embodiment, applying second ensemble model 214 to first plurality of status 216 may include detecting a second plurality of status from clinical data set 208 (step 114), detecting a third plurality of status from the second plurality of status (step 116), and applying second ensemble model 214 to the third plurality of status (step 118). Second model 214 may be applied to first plurality of status 216 utilizing an exemplary processor.

In an exemplary embodiment, step 114 may include detecting a second plurality of status. The second plurality of status may be detected utilizing an exemplary processor. In an exemplary embodiment, each of the second plurality of status may include a respective binary value representing one of the illness or the healthiness. In an exemplary embodiment, detecting the second plurality of status may include applying each of a plurality of adaptive neuro fuzzy inference systems (ANFISs) to a respective subset of clinical data set 208. In an exemplary embodiment, a status d_{2,m} of the second plurality of status may be generated by applying an ANFIS 222 of the plurality of ANFISs to i^{th} subset 220 where 1≤m≤M and M is a number of the plurality of ANFISs. An exemplary ANFIS may include a type of neural networks that is integrated with fuzzy logic principles. An exemplary ANFIS may include an intelligent neuro-fuzzy technique utilized for modeling and control of ill-defined and uncertain systems. Therefore, exemplary ANFISs may enhance a classification performance of first classifier 202 by integrating classification results of plurality of statistical processes 212 and the plurality of ANFISs. In an exemplary embodiment, answers of a subject to an EDSS test may be applied to ANFIS 222 and a respective status may be detected.

In an exemplary embodiment, step 116 may include detecting a third plurality of status. The second plurality of status may be detected utilizing an exemplary processor. In an exemplary embodiment, each of the third plurality of status may include a respective binary value representing one of the illness or the healthiness. In an exemplary embodiment, two different status may be detected by applying i^{th} subset 220 to both i^{th} statistical process 218 and ANFIS 222. In an exemplary embodiment, a respective status of the third plurality of status detected from i^{th} subset 220 may then be generated by combining two different status. In an exemplary embodiment, detecting the third plurality of status may include applying each of a plurality of ensemble models to a respective status of first plurality of status 216 and a respective status of the second plurality of status. In an exemplary embodiment, a status d_{3,m} of the third plurality of status may be detected by applying an ensemble model 224 to status d_{1,i} and status d_{2,m}. In an exemplary embodiment, when status d_{1,i} and status d_{2,m} are equal, ensemble model 224 may return a class of both status d_{1,i} and status d_{2,m}. In contrast, in an exemplary embodiment, ensemble model 224 may return a class of status d_{1,i} when status d_{1,i} and status d_{2,m} are different.

In an exemplary embodiment, step 118 may include applying second ensemble model 214 to the third plurality of status. In an exemplary embodiment, second ensemble model 214 may generate a classification result obtained by first classifier 202. Second ensemble model 214 may include an artificial neural network and may be implemented by an exemplary processor. In an exemplary embodiment, first status d₁ may be obtained by applying first plurality of status 216 to second ensemble model, that is, the artificial neural network. In other words, first status d₁ may include an output of an exemplary artificial neural network responsive to first plurality of status 216 being applied to the artificial neural network. In an exemplary embodiment, each of the third plurality of status may include one of first plurality of status 216 or one of the second plurality of status. In an exemplary embodiment, a subset 226 of clinical data set 208 may not be applied to an ANFIS. In an exemplary embodiment, a status (similar to status d_{1,1}) detected from subset 226 may be directly applied to ensemble model 214. In other words, in an exemplary embodiment, status d_{1,1} may include one of the third plurality of status. In contrast, in an exemplary embodiment, a status (similar to status d_{1,i}) of first plurality of status 216 may be applied to an ANFIS (similar to ANFIS 222). In an exemplary embodiment, an output of ANFIS 222, that is, status d_{2,m}, may be applied to ensemble model 224 and an output of ensemble model 224 may be applied to ensemble model 214. In other words, in an exemplary embodiment, an output of ensemble model 224 may include one of the third plurality of status.

An exemplary ensemble model may include a machine learning technique that combines multiple individual models to improve an overall performance and predictive accuracy of a model. An exemplary ensemble model may become more accurate and reliable than any individual model by combining a number of predictions of individual models. An exemplary model may combine predictions of individual models by applying a majority voting on the predictions. An exemplary majority voting model may be referred to as a technique used in machine learning for combining predictions of multiple individual models in an ensemble by taking a mostly predicted class by individual models as a final classification. An exemplary majority voting model may receive classification results of a number of classifiers and return a class with maximum number of classes. As a result, a classification precision may enhance because a probability of wrong classification by a majority of classifiers may be likely less than a probability of wrong classification by each of classifiers. Another exemplary voting majority model may put different weights on classification results of different classifiers, obtaining a trade-off between performances of different classifiers in terms of error variance and error bias.

An exemplary ensemble model may include a bootstrap aggregation (bagging) model. An exemplary bagging model may be implemented by splitting a training data set into a number of random subsets and training a number of individual models by applying each of the random subsets on a respective individual model. Each exemplary individual model may include one of a decision tree, a random forest, or a logistic regression model. Then, exemplary predictions of individual models may be combined by applying a majority voting model to the predictions that includes taking a class (illness or healthiness) that is predicted by most individual models as a final prediction of the bagging model.

Referring again to FIGS. 1A and 2A, in an exemplary embodiment, step 106 may include detecting a second status d₂. Second status d₂ may be detected utilizing an exemplary processor. In an exemplary embodiment, second status d₂ may be detected by applying second classifier 204 to para-clinical data set 210. Second classifier 204 may be implemented utilizing an exemplary processor.

For further detail regarding step 106, FIG. 1E shows a flowchart for applying a classifier to a para-clinical data set, consistent with one or more exemplary embodiments of the present disclosure. In an exemplary embodiment, applying second classifier 204 to para-clinical data set 210 may include detecting a fourth plurality of status from the plurality of medical images (step 120) and detecting an image status from the fourth plurality of status (step 122).

FIG. 2C shows a schematic of a second classifier, consistent with one or more exemplary embodiments of the present disclosure. In an exemplary embodiment, second classifier 204 may include a third ensemble model 228, a fourth ensemble model 230, and a fifth ensemble model 232. Specifically, in an exemplary embodiment, second classifier classifies para-clinical data set 210 into one of illness class or healthiness class of a subject by aggregating outputs of third ensemble model 228, fourth ensemble model 230, and fifth ensemble model 232. In an exemplary embodiment, each of third ensemble model 228, fourth ensemble model 230, and fifth ensemble model 232 may be implemented by a respective majority voting model.

Referring to FIGS. 1E and 2C, in an exemplary embodiment, step 120 may include detecting a fourth plurality of status 234 from a plurality of medical images 236 of para-clinical data set 210. In an exemplary embodiment, each of the fourth plurality of status may include a respective binary value representing one of the illness or the healthiness. In an exemplary embodiment, the plurality of medical images may include at least one of computed tomography (CT) scan images, magnetic resonance images (MRI), magnetic resonance venography (MRV) images, magnetic resonance spectroscopy (MRS) images, and positron-emission tomography (PET) images. An exemplary image status detected from the plurality of images may represent one of the healthiness or being subjected to an MS disease.

In an exemplary embodiment, detecting fourth plurality of status 234 may include applying a first plurality of machine learning (ML)-based classifiers 238 to plurality of medical images 236. In an exemplary embodiment, a status d_{4,n} of fourth plurality of status 234 may be detected by applying an n^{th} ML-based classifier 240 to an image set 242 of plurality of medical images 236 where 1≤n≤N₄ and N₄ is a number of fourth plurality of status 234. In an exemplary embodiment, first plurality of ML-based classifiers 238 may include a plurality of U-Nets. In an exemplary embodiment, image set 242 may include different modalities of MRIs such as T1 (spin-lattice relaxation), T2 (spin-spin relaxation), and T2-Flair (fluid attenuation inversion recovery). In an exemplary embodiment, each modality of MRIs may be applied to a respective U-Net of a number of primary U-Nets. Then, in an exemplary embodiment, outputs of primary U-Nets may be concatenated and a result of concatenation may be applied to a secondary U-Net. An exemplary output of the secondary U-net may include one of fourth plurality of status 234.

In an exemplary embodiment, step 122 may include detecting an image status d_{I} from fourth plurality of status 234. In an exemplary embodiment, detecting image status d_{I} may include applying third ensemble model 228 to fourth plurality of status 234. In an exemplary embodiment, third ensemble model 228 may include a majority voting model. Therefore, in an exemplary embodiment, third ensemble model 228 may generate image status d_{I} that includes healthiness of the subject when a majority of the plurality of U-Nets detect the healthiness of the subject. In contrast, in an exemplary embodiment, third ensemble model 228 may generate image status d_{I} that includes illness of the subject when a majority of the plurality of U-Nets detect the illness of the subject.

In an exemplary embodiment, applying second classifier 204 to para-clinical data set 210 in step 106 may further include detecting a fifth plurality of status 244 from the plurality of biomedical signals (step 124) and detecting a biomedical status d_{B} from fifth plurality of diagnosis decisions 244 (step 126).

In an exemplary embodiment, step 124 may include detecting fifth plurality of status 244. Fifth plurality of status 244 may be detected utilizing an exemplary processor. In an exemplary embodiment, each of the fifth plurality of status may include a respective binary value representing one of the illness or the healthiness. In an exemplary embodiment, detecting fifth plurality of status 244 may include applying a second plurality of ML-based classifiers 246 to a plurality of biomedical signals 248 of para-clinical data set 210. Second plurality of ML-based classifiers 246 may be implemented utilizing an exemplary processor. In an exemplary embodiment, plurality of biomedical signals 248 may include at least one of an auditory evoked potential, a somatosensory evoked potential, a visually evoked potential, an electroretinogram signal, an electroneurogram signal, an electromyogram signal, and an electroencephalography signal. An exemplary biomedical status detected from the plurality of biomedical signals 248 may represent one of the healthiness or being subjected to an MS disease.

In an exemplary embodiment, detecting fifth plurality of status 244 may include applying second plurality of ML-based classifiers 246 to plurality of biomedical signals 248. In an exemplary embodiment, a status d_{5,n} of fifth plurality of status 244 may be detected by applying an n^{th} ML-based classifier 250 to a biomedical signal 252 of plurality of biomedical signals 248 where 1≤n≤N₅ and N₅ is a number of fifth plurality of status 244. In an exemplary embodiment, second plurality of ML-based classifiers 246 may include a k-nearest neighbors (KNN) classifier, a support vector machine (SVM) classifier, and a recurrent neural network (RNN). In an exemplary embodiment, each of ML-based models, i.e., the KNN classifier, the SVM classifier, and the RNN, may be obtained by training the ML-based models based on a number of biomedical signals of labeled patients. In doing so, healthcare professionals may label a number of patients as healthy or ill, and corresponding biomedical signals of labeled patients may be used to train the ML-based models. Then, fifth plurality of status 244 may be obtained by applying trained ML-based models to plurality of biomedical signals 248.In an exemplary embodiment, step 126 may include detecting a biomedical status d_{B} from fifth plurality of diagnosis decisions 244. In an exemplary embodiment, detecting biomedical status d_{B} may include applying fourth ensemble model 230 to fifth plurality of status 244. In an exemplary embodiment, fourth ensemble model 230 may include a majority voting model. Therefore, in an exemplary embodiment, fourth ensemble model 230 may generate biomedical status d_{B} that includes healthiness of the subject when a majority of ML-based classifiers 246 detect the healthiness of the subject. In contrast, in an exemplary embodiment, fourth ensemble model 230 may generate biomedical status d_{B} that includes illness of the subject when a majority of ML-based classifiers 246 detect the illness of the subject.

In an exemplary embodiment, applying second classifier 204 to para-clinical data set 210 in step 106 may further include detecting a sixth plurality of status 254 from the plurality of para-clinical test results (step 128) and detecting a para-clinical status from sixth plurality of diagnosis decisions 254 (step 130).

In an exemplary embodiment, step 128 may include detecting sixth plurality of status 254 from a plurality of para-clinical test results 256 of para-clinical data set 210. Sixth plurality of status 254 may be detected utilizing an exemplary processor. In an exemplary embodiment, detecting sixth plurality of status 254 may include comparing plurality of para-clinical test results 256 with a plurality of threshold values. In an exemplary embodiment, plurality of threshold values may be determined by healthcare professionals and based on a type of a disease under study. In an exemplary embodiment, plurality of para-clinical test results 256 may be compared with the plurality of threshold values utilizing a plurality of comparators 258. In an exemplary embodiment, comparing plurality of para-clinical test results 256 with the plurality of threshold values may include comparing each test result of a plurality of lumbar puncture test results and a plurality of blood test results with a respective threshold value of the plurality of threshold values. In an exemplary embodiment, a test result 260 of plurality of test results 256 may be compared with a threshold value utilizing a comparator 262 of plurality of comparators 258. Plurality of comparators 258 may be implemented utilizing an exemplary processor. An exemplary test result of plurality of test results 256 may be selected based on an impacting parameter on a subject's disease. Each exemplary disease may need a number of corresponding test results specific to the disease. During an exemplary lumbar puncture test, a sample of cerebrospinal fluid (CSF) of a subject may be collected. An exemplary test result of a lumbar puncture test may include each of a color, a clarity, and a pressure of CSF during collection, protein levels, glucose levels, a cell count, and a differential cell count.

In an exemplary embodiment, step 130 may include detecting a para-clinical status d_{P} from sixth plurality of diagnosis decisions 254. Para-clinical status d_{P} may be detected utilizing an exemplary processor. In an exemplary embodiment, detecting para-clinical status d_{P} may include applying fifth ensemble model 232 to sixth plurality of diagnosis decisions 254. In an exemplary embodiment, fifth ensemble model 232 may include a majority voting model. Therefore, in an exemplary embodiment, fifth ensemble model 232 may generate para-clinical status d_{P} that includes healthiness of the subject when a majority of outputs generated by plurality of comparators 258 include the healthiness of the subject. In contrast, in an exemplary embodiment, fifth ensemble model 232 may generate para-clinical status d_{P} that includes illness of the subject when a majority of outputs generated by plurality of comparators 258 include the illness of the subject.

In an exemplary embodiment, applying second classifier 204 to para-clinical data set 210 in step 106 may further include applying a sixth ensemble model 264 to image status d_{I}, biomedical status d_{B}, and para-clinical status d_{P} (step 132). Sixth ensemble model 264 may be implemented utilizing an exemplary processor. An exemplary bootstrap aggregation model may include a first implementation of sixth ensemble model 264. An exemplary bootstrap aggregation model may include an ML-based ensemble model designed to improve stability and accuracy of a number of ML classifiers. An exemplary bootstrap aggregation model may reduce variance of a classification errors and may avoid an overfitting of ML-based classification models. In an exemplary bootstrap aggregation, a number of data sets (also called bootstraps) may be generated from a training data set by sampling the training data set with replacement. A size of exemplary bootstraps may be equal to a size of the training data set. Then, exemplary bootstraps may be applied to a number of classifiers and outputs of different classifiers may be aggregated together to generate a final classification result. In training of an exemplary bootstrap aggregation model, a training data set is required. An exemplary training data set may include a number of image status, biomedical status, and para-clinical status for a number of labeled patients, that is, patients with known disease status labeled by healthcare professionals. An exemplary training data set is decomposed into a number of bootstraps by bootstrap sampling, and a number of individual models may be trained by bootstraps. Each of individual models may include a specific type of ML-based classifiers, such as decision trees, random forests, or logistic regression. Then. an exemplary majority voting model may be applied to outcomes of individual models to predict a final classification result. After training an exemplary bootstrap aggregation model, image status d_{I}, biomedical status d_{B}, and para-clinical status d_{P} may be applied to the bootstrap aggregation model to obtain second status d₂.

For further detail regarding step 132, FIG. 1F shows a flowchart for applying a classifier to a number of status, consistent with one or more exemplary embodiments of the present disclosure. Referring to FIGS. 1F and 2C, in an exemplary embodiment, applying sixth ensemble model 264 in step 132 may include calculating a weighted average (step 134), detecting a third status (step 136), setting second status d₂ to a first decision value (step 138) responsive to the weighted average and the third status being equal to the second decision value (step 140, Yes), and setting second status d₂ to a second decision value (step 142) responsive to the weighted average and the third status being different (step 140, No). In an exemplary embodiment, operations in steps 134-140 may include a second implementation of sixth ensemble model 264.

FIG. 2D shows a schematic of an ensemble model, consistent with one or more exemplary embodiments of the present disclosure. Referring to 1F and 2D, in an exemplary embodiment, second status d₂ may be detected by applying image status d_{I}, biomedical status d_{B}, and para-clinical status d_{P} to sixth ensemble model 264. In an exemplary embodiment, sixth ensemble model 264 may be implemented utilizing an average calculator 266, a neural network-based classifier 268, and a comparator 270. Average calculator 266, neural network-based classifier 268, and comparator 270 may be implemented utilizing an exemplary processor.

In an exemplary embodiment, step 134 may include calculating a weighted average μ. In an exemplary embodiment, calculating weighted average μ may include calculating a weighted average of image status d_{I}, biomedical status d_{B}, and para-clinical status d_{P}. In an exemplary embodiment, weighted average μ may be calculated utilizing average calculator 266. In an exemplary embodiment, each of image status d_{I}, biomedical status d_{B}, and para-clinical status d_{P} may include a respective number that indicates a status. Therefore, in an exemplary embodiment, weighted average μ may be equal to a weighted average of numbers that indicate status from images, signals, and test results. In an exemplary embodiment, in calculating weighted average μ, each status may be multiplied with a respective weight. An exemplary weight of each status may represent an importance level of each type of data in health status detection. Exemplary weights may vary for different diseases. For an exemplary MS disease, a weight of image status d_{I} may be larger than biomedical status d_{B} and para-clinical status d_{P}. Exemplary weights may be determined by a number of healthcare professionals such as radiologists and neurologists.

In an exemplary embodiment, step 136 may include detecting a third status d₃. In an exemplary embodiment, detecting third status d₃ may include applying neural network-based classifier 268 to image status d_{I}, biomedical status d_{B}, and para-clinical status d_{P}. In an exemplary embodiment, neural network-based classifier 268 may include a U-Net.

In an exemplary embodiment, step 138 may include setting second status d₂ to a first decision value v₁. Second status d₂ may be set to first decision value v₁ utilizing an exemplary processor. In an exemplary embodiment, first decision value v₁ may be set to each of weighted average μ and third status d₃ responsive to weighted average μ and third status d₃ being equal. Specifically, an exemplary processor may compare weighted average μ and third status d₃, and may set first decision value v₁ to each of weighted average μ and third status d₃ responsive to weighted average μ and third status d₃ being equal. In other words, when both weighted average μ and third status d₃ include a class C₁, first decision value v₁ may be set to class C₁. In an exemplary embodiment, class C₁ may include one of a “healthiness” class or an “illness” class.

In an exemplary embodiment, step 140 may include examining a condition on weighted average μ and third status d₃. An exemplary condition may include an equality of weighted average μ and third status d₃. In an exemplary embodiment, an equality of weighted average μ and third status d₃ may be examined utilizing comparator 270. In other words, an exemplary processor may receive weighted average μ and third status d₃, and may check the equality of weighted average μ and third status d₃.

In an exemplary embodiment, step 142 may include setting second status d₂ to a second decision value v₂. Second status d₂ may be set to second decision value v₂ utilizing an exemplary processor. In doing so, an exemplary processor may compare weighted average μ and third status d₃, and may set second status d₂ to second decision value v₂ responsive to weighted average μ and third status d₃ being different. In an exemplary embodiment, weighted average μ and third status d₃ may be different. Therefore, in an exemplary embodiment, a status may not be detected from para-clinical data set 210. In other words, when weighted average μ and third status d₃ are different, second decision value v₂ may be set to a value that may not stand for none of diagnostic classes. Exemplary diagnostic classes may be represented by one for a “healthiness” class and zero for an “illness” class. Therefore, in an exemplary embodiment, second decision value v₂ may be equal to a value between zero and one, indicating that a classification result based on para-clinical data set 210 is invalid.

Referring again to FIGS. 1A and 2A, in an exemplary embodiment, step 107 may include detecting a final status d_{f}. Final status d_{f} may be detected utilizing an exemplary processor. In an exemplary embodiment, final status d_{f} may be detected by applying first ensemble model 206 to first status d₁ and second status d₂. In an exemplary embodiment, first ensemble model 206 may include a boosting method. An exemplary boosting method may generate a strong classifier, that is, a classifier with low classification error, from a number of weak classifiers, that is, classifiers with high classification error. An exemplary boosting model may build a primary model from a training data set, then generating a secondary model that corrects errors from the primary model. Successive exemplary models may then be added until a training data set is predicted perfectly or a maximum number of models are added. An exemplary training data set may be obtained from a number of patients with given first status and second status. Corresponding first status and second status for each patient may be determined by healthcare professionals according to para-clinical and clinical data of patients. Then, a base model may be trained to predict a final status for each given pair of first status and second status corresponding to each patient. An exemplary final status in training phase may be obtained from healthcare professionals. An exemplary base model may include one of a decision tree, a random forest, or logistic regression. Next, a performance of an exemplary base model on the training set may be evaluated and different weights may be assigned to training instances based on classification difficulty of each instance. Specifically, instances that are misclassified by an exemplary base model may be assigned higher weights than correctly classified instances. Afterwards, an exemplary new model may be trained on the training data set, giving more importance to misclassified instances. An exemplary base model and the new model may then be combined to generate an ensemble that makes predictions based on a combined output of the base model and the new model. An exemplary majority voting may be used to combine the base model and the new model. The above-mentioned procedure may be repeated to obtain more accurate models in subsequent iteration and repetition may be continued until classification accuracy no longer improves.

In further detail with respect to step 107, FIG. 1G shows a flowchart for applying an ensemble model to a first status and a second status, consistent with one or more exemplary embodiments of the present disclosure. In an exemplary embodiment, applying first ensemble model 206 in step 107 may include generating a third decision value based on first status d₁ and second status d₂ (step 144), setting a bias of each activation function of a multi-layer perceptron (MLP) to a first bias value (step 146) responsive to the third decision value being larger than or equal to a decision threshold (step 148, No), setting the bias to a second bias value (step 150) responsive to the third decision value being smaller than the decision threshold (step 148, Yes), and applying the MLP to the first status and the second status (step 152). First ensemble model 206 may be applied to first status d₁ and second status d₂ utilizing an exemplary processor.

FIG. 2E shows a schematic of an ensemble model detecting a final status, consistent with one or more exemplary embodiments of the present disclosure. In an exemplary embodiment, first ensemble model 206 may be implemented by a decision rule 272, a comparator 274, and an MLP 276. Decision rule 272, comparator 274, and MLP 276 may be implemented utilizing an exemplary processor.

Referring to FIGS. 1G and 2E, in an exemplary embodiment, step 144 may include generating a decision value v₃ based on first status d₁ and second status d₂. Decision value v₃ may be generated utilizing an exemplary processor. In an exemplary embodiment, generating decision value v₃ may include applying decision rule 272 to first status d₁ and second status d₂. An exemplary decision rule may be referred to a function that maps an observation to an appropriate action. In an exemplary embodiment, decision rule 272 may map first status d₁ and second status d₂ to decision value v₃. An exemplary decision rule may be determined by minimizing a loss function corresponding to a classification performance of a classifier. In an exemplary embodiment, decision rule 272 may be obtained by minimizing a classification error of method 100.

In an exemplary embodiment, step 146 may include setting a bias v_{b} of each activation function of MLP 276 to the first bias value. Bias v_{b} may be set to the first bias value utilizing an exemplary processor. An exemplary bias of activation functions of MLP 276 may impact a classification performance. An exemplary first bias value may be obtained by minimizing a classification error at an output of MLP 276 when decision value v₃ is larger than or equal to a threshold value vₜₕ.

In an exemplary embodiment, step 148 may include comparing a decision value v₃ with threshold value vₜₕ. In an exemplary embodiment, a decision value v₃ may be compared with threshold value vₜₕ utilizing comparator 274.

In an exemplary embodiment, step 150 may include setting bias v_{b} to the second bias value. An exemplary bias of activation functions of MLP 276 may impact a classification performance. An exemplary second bias value may be obtained by minimizing a classification error at an output of MLP 276 when decision value v₃ is smaller than threshold value vₜₕ.

In an exemplary embodiment, step 152 may include applying MLP 276 to first status d₁ and second status d₂. In an exemplary embodiment, applying MLP 276 may include training MLP 276. In an exemplary embodiment, final status d_{f} may be extracted from an output of MLP 276. In an exemplary embodiment, MLP 276 may be trained with a number of training clinical data sets and training para-clinical data-sets. Exemplary training clinical data sets and training para-clinical data sets may be labeled according to suggestions of healthcare professionals such as neurologists.

Referring again to FIG. 1A, in an exemplary embodiment, step 108 may include determining a treatment plan of the subject. In an exemplary embodiment, final status d_{f} may include an illness class and may show a severity of a disease. Therefore, healthcare professionals may determine an appropriate treatment plan of the subject based disease type. After a diagnosis of a chronic condition such as MS disease, a treatment plan may be determined in collaboration between the healthcare professional and the subject. Goals of the treatment plan may include managing symptoms, slowing a progression of the MS disease, and improve the subject's overall quality of life. Exemplary components of the treatment plan may vary depending on a severity and type of the MS disease, as well as the subject's specific symptoms and needs. However, an exemplary treatment plan may include disease-modifying therapies (DMTs), symptomatic management, lifestyle modifications, and follow-up care.

Exemplary DMTs may include medications that slow a progression of the MS disease and reduce a frequency and severity of relapses. An exemplary DMT may depend on several factors, including a type and severity of the MS disease, the subject's age and overall health, and potential risks and benefits of medications.

MS symptoms may vary widely and may include fatigue, muscle weakness, spasticity, tremors, bladder dysfunction, and cognitive changes, among others. Symptomatic management may include medications, such as muscle relaxants or antidepressants, as well as non-pharmacological interventions, such as physical therapy, occupational therapy, or cognitive-behavioral therapy.

Maintaining a healthy lifestyle may be necessary for managing MS symptoms and preventing complications. Lifestyle modification may include regular exercise, a healthy diet, stress management techniques, and adequate rest.

An exemplary MS disease may include a chronic condition that requires ongoing management and monitoring. Follow-up care may include regular visits with a healthcare professional, imaging tests, and laboratory tests to monitor disease progression and treatment effectiveness.

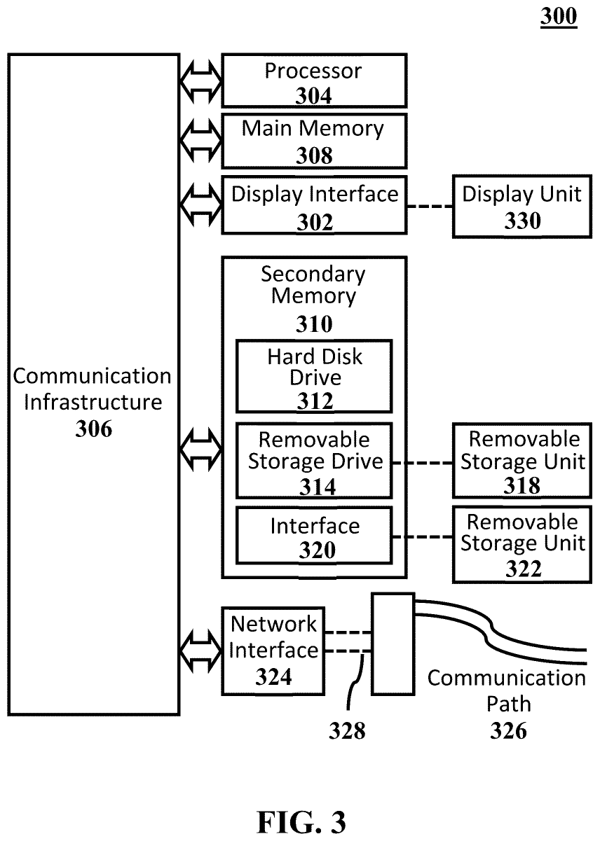

FIG. 3 shows an example computer system 300 in which an embodiment of the present invention, or portions thereof, may be implemented as computer-readable code, consistent with exemplary embodiments of the present disclosure. For example, different steps of method 100 may be implemented in computer system 300 using hardware, software, firmware, tangible computer readable media having instructions stored thereon, or a combination thereof and may be implemented in one or more computer systems or other processing systems. Hardware, software, or any combination of such may embody any of the modules and components in FIGS. 1A-2B.

If programmable logic is used, such logic may execute on a commercially available processing platform or a special purpose device. One ordinary skill in the art may appreciate that an embodiment of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device.

For instance, a computing device having at least one processor device and a memory may be used to implement the above-described embodiments. A processor device may be a single processor, a plurality of processors, or combinations thereof. Processor devices may have one or more processor “cores.”

An embodiment of the invention is described in terms of this example computer system 300. After reading this description, it will become apparent to a person skilled in the relevant art how to implement the invention using other computer systems and/or computer architectures. Although operations may be described as a sequential process, some of the operations may in fact be performed in parallel, concurrently, and/or in a distributed environment, and with program code stored locally or remotely for access by single or multi-processor machines. In addition, in some embodiments the order of operations may be rearranged without departing from the spirit of the disclosed subject matter.

Processor device 304 may be a special purpose (e.g., a graphical processing unit) or a general-purpose processor device. As will be appreciated by persons skilled in the relevant art, processor device 304 may also be a single processor in a multi-core/multiprocessor system, such system operating alone, or in a cluster of computing devices operating in a cluster or server farm. Processor device 304 may be connected to a communication infrastructure 306, for example, a bus, message queue, network, or multi-core message-passing scheme.

In an exemplary embodiment, computer system 300 may include a display interface 302, for example a video connector, to transfer data to a display unit 330, for example, a monitor. Computer system 300 may also include a main memory 308, for example, random access memory (RAM), and may also include a secondary memory 310. Secondary memory 310 may include, for example, a hard disk drive 312, and a removable storage drive 314. Removable storage drive 314 may include a floppy disk drive, a magnetic tape drive, an optical disk drive, a flash memory, or the like. Removable storage drive 314 may read from and/or write to a removable storage unit 318 in a well-known manner. Removable storage unit 318 may include a floppy disk, a magnetic tape, an optical disk, etc., which may be read by and written to by removable storage drive 314. As will be appreciated by persons skilled in the relevant art, removable storage unit 318 may include a computer usable storage medium having stored therein computer software and/or data.

In alternative implementations, secondary memory 310 may include other similar means for allowing computer programs or other instructions to be loaded into computer system 300. Such means may include, for example, a removable storage unit 322 and an interface 320. Examples of such means may include a program cartridge and cartridge interface (such as that found in video game devices), a removable memory chip (such as an EPROM, or PROM) and associated socket, and other removable storage units 322 and interfaces 320 which allow software and data to be transferred from removable storage unit 322 to computer system 300.

Computer system 300 may also include a communications interface 324. Communications interface 324 allows software and data to be transferred between computer system 300 and external devices. Communications interface 324 may include a modem, a network interface (such as an Ethernet card), a communications port, a PCMCIA slot and card, or the like. Software and data transferred via communications interface 324 may be in the form of signals, which may be electronic, electromagnetic, optical, or other signals capable of being received by communications interface 324. These signals may be provided to communications interface 324 via a communications path 326. Communications path 326 carries signals and may be implemented using wire or cable, fiber optics, a phone line, a cellular phone link, an RF link or other communications channels.

In this document, the terms “computer program medium” and “computer usable medium” are used to generally refer to media such as removable storage unit 318, removable storage unit 322, and a hard disk installed in hard disk drive 312. Computer program medium and computer usable medium may also refer to memories, such as main memory 308 and secondary memory 310, which may be memory semiconductors (e.g. DRAMs, etc.).

Computer programs (also called computer control logic) are stored in main memory 308 and/or secondary memory 310. Computer programs may also be received via communications interface 324. Such computer programs, when executed, enable computer system 300 to implement different embodiments of the present disclosure as discussed herein. In particular, the computer programs, when executed, enable processor device 304 to implement the processes of the present disclosure, such as the operations in method 100 illustrated by flowchart 100 of FIG. 1A and flowchart 102 of FIG. 1B discussed above. Accordingly, such computer programs represent controllers of computer system 300. Where an exemplary embodiment of method 100 is implemented using software, the software may be stored in a computer program product and loaded into computer system 300 using removable storage drive 314, interface 320, and hard disk drive 312, or communications interface 324.

Embodiments of the present disclosure also may be directed to computer program products including software stored on any computer useable medium. Such software, when executed in one or more data processing device, causes a data processing device to operate as described herein. An embodiment of the present disclosure may employ any computer useable or readable medium. Examples of computer useable mediums include, but are not limited to, primary storage devices (e.g., any type of random access memory), secondary storage devices (e.g., hard drives, floppy disks, CD ROMS, ZIP disks, tapes, magnetic storage devices, and optical storage devices, MEMS, nanotechnological storage device, etc.).

The embodiments have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

### EXAMPLE

In this example, a performance of a method (similar to method 100) for health status detection is demonstrated. Different steps of the method are implemented utilizing a system (similar to system 200). The method detects a status about an MS disease from magnetic resonance (MR) images (similar to plurality of medical images 236) and electroencephalography (EEG) signals (similar to plurality of biomedical signals 248).

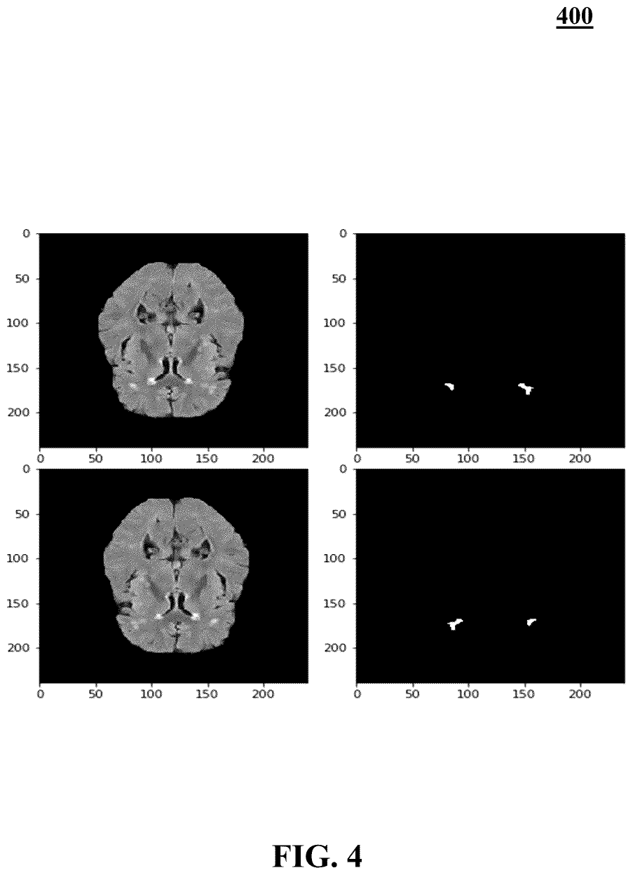

FIG. 4 shows magnetic resonance images of a subject's brain, consistent with one or more exemplary embodiments of the present disclosure. Two images of an axial view of a subject's brain (in left of FIG. 4) are fed to a U-Net (similar to ML-based classifier 240) and corresponding results (in right of FIG. 4) are extracted. As FIG. 4 shows, the U-Net highlights two lesion areas in the subject' brain. Therefore, a status (similar to status d_{4,N}_{₄}) from MR images includes an “illness” class for the corresponding subject.

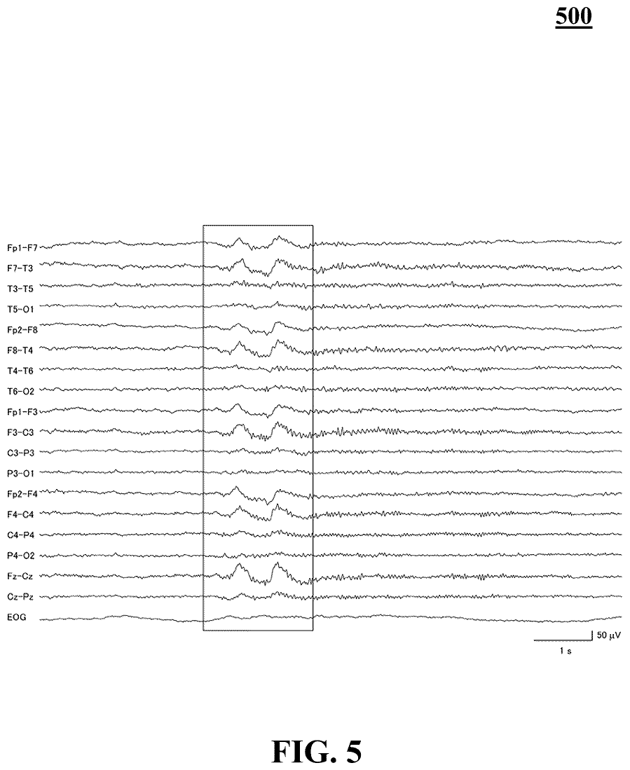

FIG. 5 shows electroencephalography signals of a subject, consistent with one or more exemplary embodiments of the present disclosure. Each row in FIG. 5 corresponds to a specific EEG channel. EEG signals (similar to plurality of biomedical signals 248) of the subject are fed to a number of recurrent neural networks (RNNs). Each RNN (similar to ML-based classifier 250) detects features of MS in a corresponding EEG signal (rectangular area in FIG. 5). Therefore, a status (similar to status d_{5,N}_{₅}) at an output of each RNN that detects MS features includes an “illness” class for the corresponding subject.

While the foregoing has described what may be considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various implementations. This is for purposes of streamlining the disclosure, and is not to be interpreted as reflecting an intention that the claimed implementations require more features than are expressly recited in each claim. Rather, as the following claims reflect, inventive subject matter lies in less than all features of a single disclosed implementation. Thus, the following claims are hereby incorporated into the Detailed Description, with each claim standing on its own as a separately claimed subject matter.

While various implementations have been described, the description is intended to be exemplary, rather than limiting and it will be apparent to those of ordinary skill in the art that many more implementations and implementations are possible that are within the scope of the implementations. Although many possible combinations of features are shown in the accompanying figures and discussed in this detailed description, many other combinations of the disclosed features are possible. Any feature of any implementation may be used in combination with or substituted for any other feature or element in any other implementation unless specifically restricted. Therefore, it will be understood that any of the features shown and/or discussed in the present disclosure may be implemented together in any suitable combination. Accordingly, the implementations are not to be restricted except in light of the attached claims and their equivalents. Also, various modifications and changes may be made within the scope of the attached claims.

What is claimed is:

1. A method for machine learning-based disease diagnosis and treatment of a subject, the method comprising:
obtaining a clinical data set associated with clinical symptoms of the subject;
obtaining a para-clinical data set by at least one of:
obtaining, utilizing a set of imaging devices, a plurality of medical images from the subject;
obtaining, utilizing a set of biomedical signal acquisition devices, a plurality of biomedical signals from the subject; and
obtaining a plurality of para-clinical test results of the subject;
detecting, utilizing one or more processors, a first status by:
detecting a first plurality of status by applying each of a plurality of statistical processes to a respective subset of the clinical data set, each subset of the clinical data set associated with a respective clinical examination; and
generating the first status by:
detecting a second plurality of status by applying each of a plurality of adaptive neuro fuzzy inference systems to a respective subset of the clinical data set;
detecting a third plurality of status by applying each of a plurality of ensemble models to a respective status of the first plurality of status and a respective status of the second plurality of status; and
applying a first ensemble model to the third plurality of status;
detecting, utilizing the one or more processors, a second status by:
detecting a fourth plurality of status by applying a first plurality of machine learning-based classifiers to the plurality of medical images;
detecting an image status by applying a first majority voting model to the fourth plurality of status;
detecting a fifth plurality of status by applying a second plurality of machine learning-based classifiers to the plurality of biomedical signals;
detecting a biomedical status by applying a second majority voting model to the fifth plurality of status;
detecting a sixth plurality of status by comparing the plurality of para-clinical test results with a plurality of threshold values; and
detecting a para-clinical status by applying a third majority voting model to the sixth plurality of status;
calculating a weighted average of the image status, the biomedical status, and the para-clinical status;
detecting a third status by applying a neural network-based classifier to the image status, the biomedical status, and the para-clinical status;
setting the second status to a first decision value responsive to each of the weighted average and the third status being equal to the first decision value; and
setting the second status to a second decision value responsive to the weighted average and the third status being different;
detecting, utilizing the one or more processors, a final status by:
generating a third decision value by applying a decision rule to the first status and the second status;
setting a bias of each activation function of a multi-layer perceptron to a first bias value responsive to the third decision value being larger than or equal to a decision threshold;
setting the bias to a second bias value responsive to the third decision value being smaller than the decision threshold; and
applying the multi-layer perceptron to the first status and the second status; and
determining a treatment plan of the subject based on the final status.

2. A method for machine learning-based disease diagnosis and treatment of a subject, the method comprising:
obtaining a clinical data set associated with clinical symptoms of the subject;
obtaining a para-clinical data set, comprising at least one of:
obtaining, utilizing a set of imaging devices, a plurality of medical images from the subject;
obtaining, utilizing a set of biomedical signal acquisition devices, a plurality of biomedical signals from the subject;
obtaining a plurality of para-clinical test results of the subject;
detecting, utilizing one or more processors, a first status by applying a first classifier to the clinical data set;
detecting, utilizing the one or more processors, a second status by applying a second classifier to the para-clinical data set;
detecting, utilizing the one or more processors, a final status by applying a first ensemble model to the first status and the second status; and
determining a treatment plan of the subject based on the final status,
wherein each of the first status, the second status, and the final status representing one of illness or healthiness of the subject.

3. The method of claim 2, wherein applying the first ensemble model comprises:
generating a first decision value by applying a decision rule to the first status and the second status;
setting a bias of each activation function of a multi-layer perceptron to a first bias value responsive to the first decision value being larger than or equal to a decision threshold;
setting the bias to a second bias value responsive to the first decision value being smaller than the decision threshold; and
applying the multi-layer perceptron to the first status and the second status.

4. The method of claim 2, wherein applying the first classifier to the clinical data set comprises:
detecting a first plurality of status by applying each of a plurality of statistical processes to a respective subset of the clinical data set, each subset of the clinical data set associated with a respective clinical examination, each of the first plurality of status comprising a respective binary value representing one of the illness or the healthiness; and
generating the first status by applying a second ensemble model to the first plurality of status.

5. The method of claim 4, wherein applying the second ensemble model to the first plurality of status comprises:
detecting a second plurality of status by applying each of a plurality of adaptive neuro fuzzy inference systems to a respective subset of the clinical data set, each of the second plurality of status comprising a respective binary value representing one of the illness or the healthiness;
detecting a third plurality of status by applying each of a plurality of ensemble models to a respective status of the first plurality of status and a respective status of the second plurality of status, each of the third plurality of status comprising a respective binary value representing one of the illness or the healthiness; and
applying the second ensemble model to the third plurality of status.

6. The method of claim 2, wherein applying the second classifier to the para-clinical data set comprises:
detecting a fourth plurality of status by applying a first plurality of machine learning-based classifiers to the plurality of medical images, each of the fourth plurality of status representing one of healthiness or illness of the subject; and
detecting an image status by applying a third ensemble model to the fourth plurality of status, the image status comprising a binary value representing one of the illness or the healthiness.

7. The method of claim 6, wherein detecting the fourth plurality of status comprises applying the first plurality of machine learning-based classifiers to the plurality of medical images by applying a plurality of U-Nets to at least one of computed tomography (CT) scan images, magnetic resonance imaging (MRI) images, magnetic resonance venography (MRV) images, magnetic resonance spectroscopy (MRS) images, and positron-emission tomography (PET) images, wherein representing the one of the healthiness or the illness comprises representing one of the healthiness or being subjected to a multiple sclerosis (MS) disease.

8. The method of claim 6, wherein applying the second classifier to the para-clinical data set further comprises:
detecting a fifth plurality of status by applying a second plurality of machine learning-based classifiers to the plurality of biomedical signals, each of the fifth plurality of status comprising a respective binary value representing one of the illness or the healthiness; and
detecting a biomedical status by applying a fourth ensemble model to the fifth plurality of status, the biomedical status comprising a binary value representing one of the illness or the healthiness.

9. The method of claim 8, wherein detecting the fifth plurality of status comprises applying the second plurality of machine learning-based classifiers to the plurality of biomedical signals by applying one of a k-nearest neighbors classifier, a support vector machine classifier, and a recurrent neural network to one of an auditory evoked potential, a somatosensory evoked potential, a visually evoked potential, an electroretinogram signal, an electroneurogram signal, an electromyogram signal, and an electroencephalography signal, wherein representing the one of the healthiness or the illness comprises representing one of the healthiness or being subjected to an MS disease.

10. The method of claim 8, wherein applying the second classifier to the para-clinical data set further comprises:
detecting a sixth plurality of status by comparing the plurality of para-clinical test results with a plurality of threshold values, each of the sixth plurality of status comprising a respective binary value representing one of the illness or the healthiness; and
detecting a para-clinical status by applying a fifth ensemble model to the sixth plurality of status, the para-clinical status comprising a binary value representing one of the illness or the healthiness.

11. The method of claim 10, wherein detecting the sixth plurality of status comprises comparing the plurality of para-clinical test results with the plurality of threshold values by comparing each test result of a plurality of lumbar puncture test results and a plurality of blood test results with a respective threshold value of the plurality of threshold values, wherein representing the one of the healthiness or the illness comprises representing one of the healthiness or being subjected to an MS disease.

12. The method of claim 10, wherein applying each of the third ensemble model, the fourth ensemble model, and the fifth ensemble model comprises applying a respective majority voting model.

13. The method of claim 10, wherein applying the second classifier to the para-clinical data set further comprises applying a bootstrap aggregation model to the image status, the biomedical status, and the para-clinical status.

14. The method of claim 10, wherein applying the second classifier to the para-clinical data set further comprises:
calculating a weighted average of the image status, the biomedical status, and the para-clinical status;
detecting a third status by applying a neural network-based classifier to the image status, the biomedical status, and the para-clinical status, the third status comprising a binary value representing one of the illness or the healthiness;
setting the second status to a second decision value responsive to each of the weighted average and the third status being equal to the second decision value; and
setting the second status to a third decision value responsive to the weighted average and the third status being different.

15. A system for machine learning-based disease diagnosis and treatment of a subject, the system comprising:
a set of para-clinical data acquisition devices, comprising at least one of:
a set of imaging devices configured to obtain a plurality of medical images of a para-clinical data set from the subject;
a set of biomedical signal acquisition devices configured to obtain a plurality of biomedical signals of the para-clinical data set from the subject;
a memory having processor-readable instructions stored therein; and
one or more processors configured to access the memory and execute the processor-readable instructions, which, when executed by the one or more processors configures the one or more processors to perform a method, the method comprising:
obtaining a clinical data set associated with clinical symptoms of the subject;
obtaining a plurality of para-clinical test results of the para-clinical data set from the subject;
detecting a first status by applying a first classifier to the clinical data set;
detecting a second status by applying a second classifier to the para-clinical data set;
detecting a final status by applying a first ensemble model to the first status and the second status; and
determining a treatment plan of the subject based on the final status,
wherein each of the first status, the second status, and the final status representing one of illness or healthiness of the subject.

16. The system of claim 15, wherein applying the first ensemble model comprises:
generating a first decision value by applying a decision rule to the first status and the second status;
setting a bias of each activation function of a multi-layer perceptron to a first bias value responsive to the first decision value being larger than or equal to a decision threshold;
setting the bias to a second bias value responsive to the first decision value being smaller than the decision threshold; and
applying the multi-layer perceptron to the first status and the second status.

17. The system of claim 15, wherein applying the first classifier to the clinical data set comprises:
detecting a first plurality of status by applying each of a plurality of statistical processes to a respective subset of the clinical data set, each subset of the clinical data set associated with a respective clinical examination, each of the first plurality of status comprising a respective binary value representing one of the illness or the healthiness; and
generating the first status by applying a second ensemble model to the first plurality of status, comprising:
detecting a second plurality of status by applying each of a plurality of adaptive neuro fuzzy inference systems to a respective subset of the clinical data set, each of the second plurality of status comprising a respective binary value representing one of the illness or the healthiness;
detecting a third plurality of status by applying each of a plurality of ensemble models to a respective status of the first plurality of status and a respective status of the second plurality of status, each of the third plurality of status comprising a respective binary value representing one of the illness or the healthiness; and
applying the second ensemble model to the third plurality of status.

18. The system of claim 15, wherein applying the second classifier to the para-clinical data set comprises:
detecting a fourth plurality of status by applying a first plurality of machine learning-based classifiers to the plurality of medical images, each of the fourth plurality of status representing one of healthiness or illness of the subject;
detecting an image status by applying a first majority voting model to the fourth plurality of status, the image status comprising a binary value representing one of the illness or the healthiness;
detecting a fifth plurality of status by applying a second plurality of machine learning-based classifiers to the plurality of biomedical signals, each of the fifth plurality of status comprising a respective binary value representing one of the illness or the healthiness;
detecting a biomedical status by applying a second majority voting model to the fifth plurality of status, the biomedical status comprising a binary value representing one of the illness or the healthiness;
detecting a sixth plurality of status by comparing the plurality of para-clinical test results with a plurality of threshold values, each of the sixth plurality of status comprising a respective binary value representing one of the illness or the healthiness;
detecting a para-clinical status by applying a third majority voting model to the sixth plurality of status, the para-clinical status comprising a binary value representing one of the illness or the healthiness; and
generating the second status from the image status, the biomedical status, and the para-clinical status.

19. The system of claim 18, wherein:
applying the first plurality of machine learning-based classifiers to the plurality of medical images comprises applying a plurality of U-Nets to at least one of computed tomography (CT) scan images, magnetic resonance imaging (MRI) images, magnetic resonance venography (MRV) images, magnetic resonance spectroscopy (MRS) images, and positron-emission tomography (PET) images;
applying the second plurality of machine learning-based classifiers to the plurality of biomedical signals comprises applying one of a k-nearest neighbors classifier, a support vector machine classifier, and a recurrent neural network to one of an auditory evoked potential, a somatosensory evoked potential, a visually evoked potential, an electroretinogram signal, an electroneurogram signal, an electromyogram signal, and an electroencephalography signal;
comparing the plurality of para-clinical test results with the plurality of threshold values comprises comparing each test result of a plurality of lumbar puncture test results and a plurality of blood test results with a respective threshold value of the plurality of threshold values; and
representing the one of the healthiness or the illness comprises representing one of the healthiness or being subjected to a multiple sclerosis (MS) disease.

20. The system of claim 18, wherein generating the second status comprises one of:
applying a bootstrap aggregation model to the image status, the biomedical status, and the para-clinical status; and
applying a third ensemble model to the image status, the biomedical status, and the para-clinical status by:
calculating a weighted average of the image status, the biomedical status, and the para-clinical status;
detecting a third status by applying a neural network-based classifier to the image status, the biomedical status, and the para-clinical status, the third status comprising a binary value representing one of the illness or the healthiness;
setting the second status to a second decision value responsive to each of the weighted average and the third status being equal to the second decision value; and
setting the second status to a third decision value responsive to the weighted average and the third status being different.
